(19) United States
(12) Patent Application Publication
(10) Pub. No.: US 2003/0077682 A1
(43) Pub. Date: Apr. 24, 2003

(54) Human tissue urokinase type plasminogen activator formulation

(76) Inventors: Paul Porwen Hung, Bryn Mawr, PA (US); Kenneth S. S. Chang, Taipei (JP); Bryan T. H. Wu, Taipei (JP); Kuo-Kuei Huang, Hsinchu (TW)

Correspondence Address: FISH & RICHARDSON PC, 225 FRANKLIN ST, BOSTON, MA, 02110, US

(21) Appl. No.: 10/236,881

(22) Filed: Sep. 5, 2002

Related U.S. Application Data
(60) Provisional application No. 60/318,173, filed on Sep. 7, 2001.

(51) Int. Cl. C12Q 1/48; C12N 9/12

(52) U.S. Cl. 435/15; 435/194

(57) ABSTRACT

This invention relates to an anhydrous arginine-free formulation including human tissue urokinase type plasminogen activator, lysine, phosphoric acid, and a non-ionic detergent, wherein the human tissue urokinase type plasminogen activator, lysine, phosphoric acid, and non-ionic detergent are in quantities of 10-60 mg, 100-700 mg, 20-100 mg, and 0.2-5 mg, respectively; or in quantities of the same relative ratio.

### RELATED APPLICATION

This application claims priority to U.S. Provisional Application Serial No. 60/318,173 filed Sep. 7, 2001, the contents of which are incorporated herein by reference.

### BACKGROUND

A plasminogen activator is a serine protease that converts a plasminogen into a plasmin, which, in turn, degrades a thrombus in a blood vessel that interrupts the flow of blood to vital organs. Collen, D. (1980) Thromb. Haemost. 43: 77-89. Thus, the plasminogen activator can be used for preventing or treating conditions in which it is desired to produce local fibrinolytic activity via the plasminogen activation. The conditions include stroke, venous thrombosis, pulmonary embolism, or deep vein and peripheral artery obstruction. See Bang, N. U. (1989) Circulation 79: 1391-1392.

### SUMMARY

In one aspect, this invention relates to an anhydrous arginine-free formulation that includes: human tissue urokinase type plasminogen activator; lysine; phosphoric acid; and a non-ionic detergent. In the formulation, the human tissue urokinase type plasminogen activator, lysine, phosphoric acid, and non-ionic detergent are in quantities of 10-60 mg (e.g., 15-60 mg or 30-40 mg), 100-700 mg (e.g., 150-500 mg or 200-350 mg), 20-100 mg (e.g., 30-80 mg or 40-60 mg), and 0.2-5 mg (e.g., 0.3-3 mg or 0.4-0.8 mg), respectively; or in quantities of the same relative ratio. One formulation of this invention includes the human tissue urokinase type plasminogen activator, lysine, phosphoric acid, and non-ionic detergent in quantities of 35 mg, 200 mg, 50 mg, and 0.5 mg, respectively; or in quantities of the same relative ratio. The non-ionic detergent can be polysorbate 20 or polysorbate 80.

In another aspect, this invention relates to an anhydrous arginine-free formulation that includes 5-20% (e.g., 8-16% or 10-14%) by weight human tissue urokinase type plasminogen activator and 55-85% (e.g., 60-80% or 65-75%) by weight lysine. The formulation can further include 15-20% by weight phosphoric acid, and 0.15-0.2% by weight a non-ionic detergent.

Human tissue urokinase type plasminogen activator is a hybrid protein of a human tissue plasminogen activator and a human urokinase plasminogen activator. It can be produced by a recombinant cell culture system. The amino acid sequence of such a hybrid protein is disclosed in U.S. Pat. Nos. 4,997,766, 4,916,071, 5,045,315, and 5,047,241. The term “human tissue urokinase type plasminogen activator” includes equivalents of the just-described hybrid protein, having a percent identity of at least 80% (e.g., 90%, 95% or 99%) and possessing essentially the same fibrinolytic activity (±10%). The “percent identity” of two amino acid sequences is determined using the algorithm of Karlin and Altschul (1990, Proc. Natl. Acad. Sci. USA 87: 2264-2268), modified as in Karlin and Altschul (1993, Proc. Natl. Acad. Sci. USA 90: 5873-5877). Such an algorithm is incorporated into the NBLAST and XBLAST programs of Altschul et al. (1990, J Mol. Biol. 215: 403-410). BLAST nucleotide searches are performed with the NBLAST program, score=100, wordlength=12. BLAST protein searches are performed with the XBLAST program, score=50, wordlength=3. Where gaps exist between two sequences, Gapped BLAST is utilized as described in Altschul et al. (1997, Nucleic Acids Res. 25: 3389-3402). When utilizing BLAST and Gapped BLAST programs, the default parameters of the respective programs (e.g., XBLAST and NBLAST) are used. See http://www.ncbi.nlm.nih.gov/.

The term “lysine” as used herein refers to either D- or L- forms of lysine. To retain electrical neutrality, the lysine in the formulation of this invention is balanced by phosphate (i.e., a counterion) from phosphoric acid. Other suitable counterions (e.g., acetate, citrate, succinate, sulfate, or carbonate) can be used.

Other features, objects, and advantages of the invention will be apparent from the description and from the claims.

### DETAILED DESCRIPTION

Human tissue urokinase type plasminogen activator is a hybrid protein of a human tissue plasminogen activator and a human urokinase plasminogen activator, two types of plasminogen activators found in human. For DNA and amino acid sequences of the human tissue plasminogen activator and the human urokinase plasminogen activator, see Pennica et al. (1983) Nature 301: 214; Ny et al. (1984) Proc. Natl. Acad. Sci. USA 81: 5355; and Verde et al. (1984) Proc. Natl. Acad. Sci. USA 81: 4727. Both of the plasminogen activators bind fibrin and act at the site of a thrombus. The hybrid protein is also fibrinolytically active and offers the advantages of increased stability, increased binding affinity for fibrin, and improved half-life in vivo, compared to either of the human tissue plasminogen activator or the human urokinase plasminogen. See U.S. Pat. Nos. 4,997,766, 4,916,071, 5,045,315, and 5,047,241. A formulation of this invention, which is arginine free, unexpectedly retains these properties of the human tissue urokinase type plasminogen activator.

The human tissue urokinase type plasminogen activator can be prepared by procedures known in the art. More specifically, it can be obtained from a cultured transformed cell line using recombinant DNA technology as described in U.S. Pat. Nos. 4,997,766, 4,916,071, 5,045,315, and 5,047,241. The human tissue urokinase type plasminogen activator can then be purified by column chromatography or other techniques. Purity can be readily measured by any appropriate method, for example, column chromatography, polyacryamide gel electrophoresis, high-pressure liquid chromatography analysis, or analysis of fibrinolytic activity.

One can prepare the formulation of this invention by employing the just-described human tissue urokinase type plasminogen activator in a buffer exchanging method (e.g., gel filtration or dialysis) and lyophilizing a human tissue urokinase type plasminogen activator-containing solution. A buffer (i.e., a formulation buffer) that can be used in the buffer exchanging method includes 0.1-0.7 M lysine (e.g., 0.15-0.5 M, or 0.2-0.35 M). The pH of the buffer is from 6.5 to 7.5. Additionally, the buffer includes one or more non-ionic detergents, such as polysorbate 20 or polysorbate 80, in amounts of 0.001% to 1%. After buffer exchange, e.g., dialysis, the human tissue urokinase type plasminogen activator-containing solution can be transferred to a glass vial and lypophilized to a storage form. Lyophilization, or freeze-drying, of a human tissue urokinase type plasminogen activator-containing solution can be carried out using procedures and equipments well known to those skilled in the art.

The fibrinolytic activity of the human tissue urokinase type plasminogen activator used to practice this invention is 30,000-38,000 IU/mg, e.g., 36,000 IU/mg. The fibrinolytic activity of the human tissue urokinase type plasminogen activator can be determined by a method described in, for example, U.S. Pat. No. 4,777,043. The anhydrous formulation of this invention includes a pharmaceutically effective amount of the human tissue urokinase type plasminogen activator. The pharmaceutically effective amount refers to the amount of the human tissue urokinase type plasminogen activator that provides therapeutic effect on the treated subject, such as 10-60 mg. The effective amount will also vary, as recognized by those skilled in the art, depending on the excipient usage, route of administration, and the possibility of co-usage with other therapeutic treatment.

The anhydrous formulation of this invention can be administrated to a subject utilizing conventional methods. The administration can be via the parenteral route by various injection or infusion techniques. In any event, the anhydrous formulation is dissolved in a suitable aqueous solvent, e.g., water for injection. A suitable volume of the aqueous solvent (e.g., 5 mL or 10 mL of water for injection) is needed to dissolve all components in the anhydrous formulation. As an example to prepare an appropriate dosage form, 10 mL water for injection is added to a vial containing 35 mg human tissue urokinase type plasminogen activator, 200 mg lysine, 50 mg phosphoric acid, and 0.5 mg polysorbate 80, thereby reconstituting a solution containing the human tissue urokinase type plasminogen activator. Preferably, an anhydrous formulation is used for single intravenous bolus administration immediately after reconstitution with 10 mL water for injection.

Without further elaboration, it is believed that the above description has adequately enabled the present invention. The following specific embodiments are, therefore, to be construed as merely illustrative, and not limitative of the remainder of the disclosure in any way whatsoever. All of the publications cited herein, including patents, are hereby incorporated by reference in their entirety.

Preparation of an Anhydrous Formulation

A transformed C127 hybrid human tissue type urokinase plasminogen activator-producing cell line was obtained as described in, e.g., U.S. Pat. No. 4,916,071. Cells were maintained in a DMEM:F12 (1:1) growth medium (GIBCO/BRL) supplemented with fetal bovine serum (10%), glutamine (4 mM), and gentamicin (50 μg/mL). The cell culture was incubated for one day in a humidified 37° C., 5% CO₂ incubator. Then the cells were collected and washed with 10 mL phosphate buffered saline (PBS) buffer. A 4 mL solution containing trypsin-EDTA was added to detach the cells, and the cells were transferred to 5 mL of the growth medium. The obtained cell culture was incubated at 37° C. on a roller drum with a rotation speed of 0.5 rpm. After two days, the culture solution was replaced with a fresh DMEM:F12 (1:1) growth medium containing aprotinin (10 KIU/mL), and the culture solution containing human tissue urokinase type plasminogen activator was kept. Continuously, the replacement was repeated every two days. The condition mediums were pooled and filtered sequentially through 3.0 and 0.22 μm filters.

The filtered solution was applied to a zinc chelating-Sepharose column (Pharmacia, 5.0 cm×7.5 cm). After washed with 150 mL of a PBS/TW buffer (0.02 M sodium phosphate, 0.15 M NaCl, 0.01% polysorbate 80, pH 7.4) and 600 mL of a PB buffer (0.02 M sodium phosphate, 0.3 M NaCl, 0.01% polysorbate 80, pH 7.4), the column was eluted with an elution buffer (0.02 M sodium phosphate, 0.3 M NaCl, 0.05 M imidazole, 0.01% polysorbate 80, pH 7.4). The fractions containing human tissue urokinase type plasminogen activator were collected and pooled. The pooled fractions were applied to a L-lysine Sepharose column (1.5 cm×20 cm). After washed with 35 mL of the PB buffer and 250 mL of a wash buffer (0.05 M Tris-HCl, 0.3 M NaCl, 0.01% polysorbate 80, pH 7.5), the column was eluted with another elution buffer (0.05 M Tris-HCl, 0.5 M L-Arginine, 0.3 M NaCl, 0.01% polysorbate 80, pH 7.5). Purified human tissue urokinase type plasminogen activator in the buffer was obtained. All the above-described steps were carried out at 5-8° C.

0.5 mL of the purified human tissue urokinase type plasminogen activator in the buffer was loaded into a dialysis membrane (Spectrum), and the membrane was placed in a dialysate reservoir containing 0.5 L of a formulation buffer (0.2 M lysine, 0.1 M phosphoric acid, 0.01% polysorbate 80, pH 7.1). Dialysis was performed for 18 hr at 4° C., and was continued for 18 hr after the formulation buffer was replaced with a 1 L fresh buffer, then for another 16 hr after supplied with another 1 L fresh buffer. Removed from the membrane, an aqueous solution containing human tissue urokinase type plasminogen activator was obtained and lyophilized to produce an anhydrous formulation.

Determination of a Human Tissue Urokinase Plasminogen Activator Activity

The measurement of human tissue urokinase type plasminogen activator activity was carried out by a plate fibrinolysis assay method using human tissue plasminogen activator as a standard (purchased from the NIBSC organization, labeled as tissue plasminogen activator, human, recombinant. Third International Standard 98/714). The potency of the standard was determined by an International Collaborative Study and found to be 10,000 IU/ampoule. A 15 μL thrombin solution (0.5 U/μL) was diluted with a 5 mL PBS buffer, and mixed with a 5 mL plasminogen-containing (10 U) PBS buffer. 4.5 mL of the mixed solution and 4.5 mL fibrinogen solution were added to a 9 mL of 1% aliquot agarose at 48° C. The mixture thus obtained was stirred and poured on a 90×90 mm² plate. 3 mm holes were punched on the cooled plate to make wells. Serial dilutions of standard human tissue plasminogen activator with a PBS buffer (0.1 mg/mL bovine serum albumin, 0.01% polysorbate 80) were used. 10 μL samples of the human tissue urokinase type plasminogen activator sample in the formulation buffer obtained as described above, and the standard human tissue plasminogen activator dilutions were loaded onto the wells, and incubated for 20-24 hr at room temperature. A diameter of a clear zone of fibrin was measured for each sample. For the standard human tissue plasminogen activator dilutions, the diameters of zones were plotted against activity units of dilutions, and a standard curve was obtained. For the human tissue urokinase type plasminogen activator, the diameter of zone was used to determine the activity of the human tissue urokinase type plasminogen activator sample.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, a lysine substitute with the same functional groups and pKa can be used in the formulation to practice the invention. Thus, other embodiments are also within the claims.

What is claimed is:

1. An anhydrous arginine-free formulation comprising:
human tissue urokinase type plasminogen activator;
lysine;
phosphoric acid; and
a non-ionic detergent;
wherein the human tissue urokinase type plasminogen activator, lysine, phosphoric acid, and non-ionic detergent are in quantities of 10-60 mg, 100-700 mg, 20-100 mg, and 0.2-5 mg, respectively; or in quantities of the same relative ratio.

2. The formulation of claim 1, wherein the human tissue urokinase type plasminogen activator, lysine, phosphoric acid, and non-ionic detergent are in quantities of 15-60 mg, 150-500 mg, 30-80 mg, and 0.3-3 mg, respectively; or in quantities of the same relative ratio.

3. The formulation of claim 2, wherein the non-ionic detergent is polysorbate 20.

4. The formulation of claim 2, wherein the non-ionic detergent is polysorbate 80.

5. The formulation of claim 1, wherein the human tissue urokinase type plasminogen activator, lysine, phosphoric acid, and non-ionic detergent are in quantities of 30-40 mg, 200-350 mg, 40-60 mg, and 0.4-0.8 mg, respectively; or in quantities of the same relative ratio.

6. The formulation of claim 5, wherein the non-ionic detergent is polysorbate 20.

7. The formulation of claim 5, wherein the non-ionic detergent is polysorbate 80.

8. The formulation of claim 1, wherein the human tissue urokinase type plasminogen activator, lysine, phosphoric acid, and non-ionic detergent are in quantities of 35 mg, 200 mg, 50 mg, and 0.5 mg, respectively; or in quantities of the same relative ratio.

9. The formulation of claim 8, wherein the non-ionic detergent is polysorbate 20.

10. The formulation of claim 8, wherein the non-ionic detergent is polysorbate 80.

11. The formulation of claim 1, wherein the non-ionic detergent is polysorbate 20.

12. The formulation of claim 1, wherein the non-ionic detergent is polysorbate 80.

13. An anhydrous arginine-free formulation comprising 5-20% by weight human tissue urokinase type plasminogen activator and 55-85% by weight lysine.

14. The formulation of claim 13, wherein the formulation comprises 8-16% by weight human tissue urokinase type plasminogen activator and 60-80% by weight lysine.

15. The formulation of claim 14, further comprising 15-20% by weight phosphoric acid.

16. The formulation of claim 15, further comprising 0.15-0.2% by weight a non-ionic detergent.

17. The formulation of claim 14, further comprising 0.15-0.2% by weight a non-ionic detergent.

18. The formulation of claim 13, wherein the formulation comprises 10-14% by weight human tissue urokinase type plasminogen activator and 65-75% by weight lysine.

19. The formulation of claim 18, further comprising 15-20% by weight phosphoric acid.

20. The formulation of claim 19, further comprising 0.15-0.2% by weight a non-ionic detergent.

21. The formulation of claim 18, further comprising 0.15-0.2% by weight a non-ionic detergent.

22. The formulation of claim 13, further comprising 15-20% by weight phosphoric acid.

23. The formulation of claim 22, further comprising 0.15-0.2% by weight a non-ionic detergent.

24. The formulation of claim 13, further comprising 0.15-0.2% by weight a non-ionic detergent.
